(19) United States
(12) Patent Application Publication
(10) Pub. No.: US 2024/0376431 A1
(43) Pub. Date: Nov. 14, 2024

(54) MICROBIAL CROSS-REACTIVE ANTIGENS FOR USE IN THE STIMULATION OF T-CELLS

(71) Applicants: International N&H Denmark ApS, Kongens Lyngby (DK); DANISCO US INC., Palo Alto, CA (US); DANISCO DEUTSCHLAND GMBH, Niebull (DE); NUTRITION & BIOSCIENCES USA 4, INC., Rochester, NY (US)

(72) Inventors: Wesley William Morovic, Edgerton, WI (US); Jonathan David Peterson, Egg Harbor Township, NJ (US); Charles Ryan Budinoff, Oregon, WI (US); Sebastien Guery, Munich (DE)

(21) Appl. No.: 18/687,459

(22) PCT Filed: Sep. 2, 2022
(86) PCT No.: PCT/US22/75890

§ 371 Date: Feb. 28, 2024

Related U.S. Application Data
(60) Provisional application No. 63/240,602, filed on Sep. 3, 2021.

(51) Int. Cl. C12N 5/0783; C07K 14/005

(52) U.S. Cl. CPC C12N 5/0638; C07K 14/005; C12N 2501/998; C12N 2770/20022

(57) ABSTRACT

Provided herein, inter alia, are microbial cross-reactive antigens (mCRAGs) and compositions comprising the same for use in the stimulation of T cells for the treatment or prevention of disease associated with coronavirus infection.

### CROSS-REFERENCE TO RELATED APPLICATIONS

This application is a U.S. National Stage Application under 35 U.S.C. § 371 of International Patent Application No. PCT/US2022/075890, filed Sep. 2, 2022, which claims priority to U.S. Provisional Patent Application No. 63/240,602, filed Sep. 3, 2021, the disclosure of each of which are incorporated by reference herein in their entirety.

### INCORPORATION BY REFERENCE

The Sequence Listing submitted in an XML file, in accordance with 37 C.F.R. §§ 2412 is incorporated herein by reference. The xml file name is “20240214 NB42036USPCT Seg List.xml”, the date of creation of the xml file is Feb. 14, 2024, and the size of the xml file in bytes is 164,451.

### FIELD OF THE INVENTION

This invention relates to new uses and compositions comprising polypeptides for stimulating T cells to prevent or treat illness and/or symptoms associated with coronaviruses. In particular, the coronavirus is SARS-CoV-2.

### BACKGROUND

Coronaviruses (Coronaviridae) have been long recognized as one of the causative agents of common cold and respiratory infections in humans and a variety of respiratory illnesses in animals. However, it has only been in the 21st century that coronavirus variants have emerged as pandemic pathogens. In 2002, the SARS-CoV virus emerged and in short order demonstrated the high infectivity characteristic of modern strains. The appearance of MERS coronavirus on the Arabian Peninsula followed a similar trajectory.

Since the outbreak of SARS (Severe Acute Respiratory Syndrome) coronavirus 2 (SARS-CoV-2), causing coronavirus disease (COVID-19), the world has experienced a fast spread of this highly infectious virus leading to a global pandemic. While approximately 15-20% of the tested cases are asymptomatic, most of the affected patients have mild symptoms that includes fever (83-98% of symptomatic cases), cough (59-82% of symptomatic cases), shortness of breath (19-55% of symptomatic cases) and muscle ache (11-44% of symptomatic cases). However, in some patients, this disease will progress to a more severe state that will develop around 8 days after the infection has occurred. The main symptoms go from dyspnea to respiratory distress with 3-29% of patients needing admission to Intensive Care Units (ICUs). The disease course then may lead to acute respiratory distress syndrome (ARDS) (17-29% of patients hospitalized), severe sepsis with shock and in some cases multiple organ dysfunction within one week. Last, it has been estimated that the global mortality rate of infected patients is approximately 5-7%.

The challenges presented by this virus are various: its high contagiousness combined with a fair share of asymptomatic carriers allow for the infection to spread rapidly and undetected amongst the population. This leads to a fast increase of cases in all countries infected which has put huge pressure on existing healthcare infrastructures leading to high death tolls.

Furthermore, unlike the other coronaviruses, this variant has retained its virulence even though as an RNA virus, it is expected to undergo mutation at a relatively high frequency. In addition, the lack of animal models of the disease has hindered the ability of vaccine developers to demonstrate efficacy and the rapid spread has called for employing shortcuts to get to human trials without a wealth of animal data.

Consequently, all infected countries, except a handful of them, went into extensive lockdowns, limiting social gathering, restricting travels and forcing most businesses to limit activities to its bare minimum and in most cases to close. Such measures have triggered the “economics of stoppage” creating a global recession with devastating consequences for the global population.

Over the past few months, government, non-governmental organizations (NGOs) and private firms have refocused some of their efforts to find treatments against COVID-19. The most common approaches can be clustered into three main categories: i) passive immunity/neutralizing antibodies, ii) vaccines and iii) drug repurposing.

With regards to the approaches above, one product has been approved to treat severe cases of COVID-19 (the small molecule drug Remdesivir). However, recently published clinical data do not show a strong benefit vs. placebo. On 12th of May 2020, 1368 clinical trials against COVID-19 were registered in clinicaltrials.gov

### SUMMARY OF THE INVENTION

In order to overcome the challenges presented by the current outbreak of SARS-CoV-2 and other coronavirus caused/associated respiratory illness or disease, the inventors believe that a key to a successful outcome for preventing or treating the population against coronavirus associated severe respiratory syndrome disease in general and COVID-19 specifically will be to provide a product that would: (i) enable the immune system to (a) secrete antibodies against SARS-CoV-2, (b) stimulate pre-existing SARS-CoV-2 crossreactive T cells, and (3) develop novel SARS-CoV-2 cross-reactive T cells using bacterial strains having microbial cross reactive antigens (mCRAGs) and/or (ii) activate the immune system to stimulate anti-viral immunity before infection with SARS-CoV2. The present inventors believe that this approach, i.e., the use of mCRAGs to stimulate T cells can be used alone and/or in addition to other approaches being considered currently by other research groups, like vaccines.

It is therefore an object of the present invention to provide a method and compositions comprising mCRAGs, to be used in preventing or treating illness and/or symptoms associated with coronaviruses via the stimulation of T cells. In particular, it is the object of the present invention to provide mCRAGs to be used in preventing or treating illness and/or symptoms associated with a SARS-CoV-2 virus in a subject in need thereof.

Accordingly, in some aspects, provided herein is a method for stimulating a T cell, comprising contacting the T cell with one or more polypeptide(s) comprising SEQ ID NOs. 1-71, wherein stimulating the T cell results in one or more of i) increased T cell proliferation; ii) secretion of cytokines; and iii) upregulation of one or more surface-expressed activation markers. In some embodiments, the cytokine is one or more cytokine selected from the group consisting of IFNγ, TNFα, IL2, IL17, IL22, IL4, and IL5. In some embodiments of any of the embodiments disclosed herein, the surface-expressed activation marker is one or more marker selected from the group consisting of CD69, CD134, CD137, CD154, and CD25. In some embodiments of any of the embodiments disclosed herein, the T-cell is a cytotoxic T cell, a helper T cell, and/or a γδ T cell.

### BRIEF DESCRIPTION OF THE DRAWINGS

FIG. 1 is a series of graphs depicting CD4 T-cell intracellular IFN-γ response to synthetic peptide pools measured by flow cytometry. PBMCs from 5 individual donors split into 2 fractions and treated with CRAG or Control peptide pools in a 2 week ex-vivo amplification step followed by restimulation with CRAG, Control, or SARS-CoV-2 peptide pools. Plots represent individual donor percentage of total CD4 cells expressing intracellular IFN-γ. with background DMSO vehicle control measurements subtracted. Individual charts are arranged by final peptide pool re-stimulation treatment and grouped by initial stimulation fraction. Statistical analysis was done using a ratio paired T-test.

### DETAILED DESCRIPTION

The detailed aspects of this invention are set out below. In part some of the detailed aspects are discussed in separate sections. This is for ease of reference and is in no way limiting. All of the embodiments described below are equally applicable to all aspects of the present invention unless the context specifically dictates otherwise.

Mucosal IgA is broadly cross-reactive against microbiota and helps maintain microbiota homeostasis with the host. Without being bound to theory, by stimulating T-cells with mCRAGs that have homologous epitopes with coronaviruses, including SARS-CoV-2, surface polypeptides (mainly S protein), it could be possible to induce cross-reactive IgA antibodies that could reduce the risk of infection by cross-reacting with SARS-CoV-2 or other coronavirus virions at the mucosal surfaces. Mucosal IgA antibodies also function in transporting viral particles from host side of the epithelium to microbiota side via polymeric Ig receptor expressed in epithelial cells.

To produce antigen specific reactions and novel antibody production, mCRAGs should induce T-cell dependent reactions, for example, in the gut/mucosa. If consumed as powder, these antibodies may also be produced in the mucosa of respiratory tract.

The antigen specific T-cell dependent reactions are driven in secondary lymphoid tissues (lymph nodes/peyer's patches) by (i) antigen presenting cells that ingest foreign antigens and display cross-reactive peptides on MHC-II/MHC-I molecules on their surface, (ii) CD4 T-cells recognizing the peptide: MHC-II complexes, (iii) antigen specific CD8 T-cells activated by CD4 T-cells, and (iv) B-cells activated by B-cell receptor binding the pathogen and displaying the peptide in MHC-II and (v) CD4 T-cells activated by the peptide from the same foreign antigen by antigen presenting cell.

T-cell dependent IgA production is driven by TGF-β (IgA class switching), and expansion/differentiation of B-cells by IL-5, IL-6, IL-10, and IL-21-secreted by CD4 T-cells. This is basically a tolerogenic Treg/barrier protecting Th17 response to microbiota that is induced by dendritic cells secreting IL-6, IL-10, IL-23, TGF-β.

Stimulation of T cells by mCRAGs induces IgA responses in the intestine. It has been shown that some plasma B-cells (Ab producing cells) travel to other mucosal sites (˜10%), for example, the respiratory tract (˜1-2%), to add to existing plasma cell/IgA pool. CD8 memory cells are important in eradication of virus infected cells and driving anti-viral immunity. It has been shown that healthy humans have memory CD8 T-cells against commensal microbes. Further it was shown that the bacterial strain Bifidobacterium breve harboring a cross-reactive peptide to tumor neoantigen was able to influence tumor reactive CD8 T-cells (Bessell C A, Isser A, Havel J J, Lec S, Bell D R, Hickey J W, et al. Commensal bacteria stimulate antitumor responses via T cell cross-reactivity; JCI Insight; 2020; 5 (8)), suggesting that microbiota could also influence antigen specific memory CD8 T-cell pool against pathogens. CD8 responses are typically initiated by IL-12/IFN-γ and Th1 type dendritic/CD4 T-cells. Thus, mCRAGs derived from probiotics could influence pre-existing coronavirus cross-reactive memory CD8 T cells, but also memory CD4 T-cells.

mCRAG stimulation of T cells can also influence existing plasma cell activation and IgA pool. If there is existing cross-reactive IgA against SARS-CoV-2, mCRAGs could support total IgA production by T-cell independent mechanisms. Commensal microbiota/probiotics induce total IgA production (antigen independent) in the intestine, and potentially may have an influence in extra-intestinal IgA production as well. For example, short chain fatty acids and induction of TGF-β (IgA class-switching), IL-6, retinoic acid, BAFF and APRIL from epithelia drive the IgA production.) Without being bound to theory, it is believed that mCRAGs derived from such microbiota/probiotics would result in similar induction of total and extra-intestinal IgA production as well.

For example, probiotic supplementation against cold and flu has been studied in clinical studies over 20 years. Several meta-analyses of clinical studies have shown that probiotics in general could be effective in reducing the risk and duration of the respiratory tract infections. However, there are strain specific differences between probiotics on immune stimulation and beneficial effects against respiratory tract infections. Research on B. lactis B1-04, L. acidophilus NCFM, and B. lactis Bi-07 and other studies in the literature indicate that probiotics function by “training” innate immune responses, i.e., function by priming the immune system before the viral infection. Studies have shown increased expression of interferons and innate immune cytokines prior to reducing the viral load or the risk of infections by roughly 20%.

Although all viruses have different pathogenesis and life cycle, they still induce similar anti-viral immune responses—characterized by NK-cell, ILC1, cytotoxic T lymphocyte, Th1 responses, and IgG antibody production as well as production of interferons alpha, beta, gamma, and lambda, activation of the inflammasome and Th1 associated cytokines, such as IL-12, and IP-10. Thus, stimulation of the innate immune system against SARS-CoV-2 could be effective also against other coronaviruses.

Viruses evade these immune responses by for example producing molecules that inhibit interferon production and cellular immunity. It has been reported that in SARS-CoV-2 infections interferon responses are delayed.

Probiotics, like Lactobacillus acidophilus NCFM, have been shown to induce specific pathways associated with anti-viral immunity like interferon beta, and to increase expression of receptors (TLR3) that detect viral RNA. Further, NCFM reduced the incidence of cold symptoms in children aged 3-5 years. It also drives IL-12 production in vitro. Without being bound to theory, it is believed that by selecting mCRAGs derived from one or more bacterial strain or a consortium of one or more optimized probiotics that drive/stimulate anti-viral responses, the risk of SARS-CoV-2 infection and severe COVID-19 disease can be reduced and the duration and course of the disease shortened with potential reduction in the total symptom load.

Coronaviruses can cause various illnesses or diseases in mammals and birds. In humans, these viruses can cause respiratory tract infections that can range from mild to lethal. Mild illnesses include some cases of the common cold while more lethal virus can cause severe acute respiratory syndrome (SARS) (SARS-CoV), Middle East respiratory syndrome (MERS) (MERS-CoV) and acute respiratory distress syndrome (ARDS) in the case of COVID-19 (SARS-CoV-2). In many patients, the respiratory distress is followed by severe sepsis with shock and in some cases multiple organ dysfunction within one week, resulting in a mortality rate of infected patients of approximately 5-7%.

In one embodiment, the illness caused by coronaviruses is a respiratory illness. More particularly, the respiratory illness is acute respiratory distress syndrome (ARDS).

In another embodiment, the respiratory illness is pneumonia.

According to the World Health Organization (WHO), the most common symptoms of COVID-19 are fever, dry cough and tiredness. Less common symptoms include aches and pains, sore throat, diarrhoea, conjunctivitis, headache loss of taste or smell, a rash on skin, or discolouration of fingers or toes. Serious symptoms include difficulty breathing or shortness of breath, chest pain or pressure and loss of speech or movement.

In one embodiment, the symptoms caused by coronaviruses are one or more of cough, fever, shortness of breath or difficulty breathing (dyspnea), fatigue, muscle or body aches, nausea or vomiting, diarrhea, loss or change of sense of smell (anosmia), and loss or change of sense of taste (ageusia).

In another embodiment, the prevention and/or treatment of the illness and/or symptoms associated with coronaviruses is achieved by stimulation of the immune system in the subject when in contact with one or more of the bacterial strains object of the present invention.

Coronaviruses are a group of related RNA viruses and these include 229E (alpha coronavirus), NL63 (alpha coronavirus), OC43 (beta coronavirus), HKU1 (beta coronavirus), MERS-COV (Middle East Respiratory Syndrome coronavirus), SARS-CoV, and SARS-CoV-2 virus.

In one embodiment, the present invention relates to any coronavirus belonging to the family Coronaviridae. The coronavirus are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface.

In one embodiment, the coronavirus according to the present invention is selected from the group consisting of 229E (alpha coronavirus), NL63 (alpha coronavirus), OC43 (beta coronavirus), HKU1 (beta coronavirus), MERS-COV (Middle East Respiratory Syndrome coronavirus), SARS-CoV, and SARS-CoV-2 virus.

In a particular embodiment of the present invention, the coronavirus is SARS-CoV-2 virus.

Some people are at high risk from coronavirus (clinically extremely vulnerable). These include people who have had an organ transplant; people who are having chemotherapy or antibody treatment for cancer, including immunotherapy; people who are having an intense course of radiotherapy (radical radiotherapy) for lung cancer; people who are having targeted cancer treatments that can affect the immune system (such as protein kinase inhibitors or PARP inhibitors); people who have blood or bone marrow cancer (such as leukemia, lymphoma or myeloma); people who have had a recent bone marrow or stem cell transplant, or are still taking immunosuppressant medicine; people who have a severe lung condition (such as cystic fibrosis, severe asthma or severe COPD); people who have a medical condition that means they have a very high risk of getting infections, such as SCID or sickle cell; people who are taking medicine that makes them much more likely to get infections, such as high doses of steroids or immunosuppressant medicine; and people who have a serious heart condition and are pregnant.

Some people are at moderate risk from coronavirus (clinically vulnerable). These include people who are 70 or older; people who have a lung condition that's not severe (such as asthma, COPD, emphysema or bronchitis); people who have heart disease (such as heart failure); people who have diabetes (type I or type II diabetes); people who have chronic kidney disease; people who have liver disease (such as hepatitis); people who have a condition affecting the brain or nerves (such as Parkinson's disease, motor neurone disease, multiple sclerosis or cerebral palsy); people who have a condition that means they have a high risk of getting infections; people who are taking medicine that can affect the immune system (such as low doses of steroids); people who are very obese (a BMI of 40 or above); people who are pregnant.

There are also other factors that can affect your risk, such as being male, living in a care home (such as a nursing home or long-term care facility) or being an inmate in a prison or jail.

In one embodiment, the present invention relates to a subject who has one or more pre-existing conditions selected from the group consisting of obesity, type II diabetes, chronic lung disease or moderate to severe asthma, heart conditions, immunocompromised, chronic kidney disease and liver disease.

In a further embodiment, the present invention relates to a subject who is 65 years of age or older and/or is a resident in a nursing home or long-term care facility or jail or prison.

### Methods

Provided herein are methods for stimulating a T cell, comprising contacting the T cell with one or more polypeptide(s) comprising SEQ ID NOs. 1-71, wherein stimulating the T cell results in one or more of i) increased T cell proliferation; ii) secretion of cytokines; and iii) upregulation of one or more surface-expressed activation markers.

In some embodiments, contacting the T cell with one or more (such as 1, 2, 3, 4, 5, 6, 7, 8, 9, 10, 11, 12, 13, 14, 15, or more) polypeptide(s) comprising SEQ ID NOs. 1-71 (such as one or more of SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO: 12, SEQ ID NO: 13, SEQ ID NO: 14, SEQ ID NO: 15, SEQ ID NO:16, SEQ ID NO:17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO: 29, SEQ ID NO:30, SEQ ID NO:31, SEQ ID NO:32, SEQ ID NO:33, SEQ ID NO:34, SEQ ID NO: 35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46, SEQ ID NO:47, SEQ ID NO:48, SEQ ID NO:49, SEQ ID NO:50, SEQ ID NO:51, SEQ ID NO: 52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO: 63, SEQ ID NO:64, SEQ ID NO:65, SEQ ID NO: 66, SEQ ID NO:67, SEQ ID NO:68, SEQ ID NO: 69, SEQ ID NO: 70 and/or SEQ ID NO:71), results in increased T cell proliferation. T cell proliferation can be increased by any of about 5, 10, 15, 20, 25, 30, 35, 40, 45, 50, 55, 60, 65, 70, 75, 80, 85, 90, 95, 100, 110, 120, 130, 140 or 150% or more (inclusive of all percentages falling in between these values) compared to the amount of proliferation observed in T cells that are not contacted with one or more polypeptide(s) comprising SEQ ID NOs. 1-71. Methods to measure T cell proliferation are well known in the art. In some embodiments, the T cell is a cytotoxic T cell, a helper T cell, and/or a γδ T cell.

In some embodiments, contacting the T cell with one or more (such as 1, 2, 3, 4, 5, 6, 7, 8, 9, 10, 11, 12, 13, 14, 15, or more) polypeptide(s) comprising SEQ ID NOs. 1-71 (such as one or more of SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO: 12, SEQ ID NO:13, SEQ ID NO:14, SEQ ID NO:15, SEQ ID NO:16, SEQ ID NO:17, SEQ ID NO: 18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO: 29, SEQ ID NO:30, SEQ ID NO:31, SEQ ID NO:32, SEQ ID NO:33, SEQ ID NO:34, SEQ ID NO: 35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46, SEQ ID NO:47, SEQ ID NO:48, SEQ ID NO:49, SEQ ID NO:50, SEQ ID NO:51, SEQ ID NO: 52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO: 63, SEQ ID NO:64, SEQ ID NO:65, SEQ ID NO:66, SEQ ID NO:67, SEQ ID NO:68, SEQ ID NO: 69, SEQ ID NO: 70 and/or SEQ ID NO:71), results in secretion of one or more cytokines such as, without limitation, IFNγ, TNFα, IL2, IL17, IL22, IL4, and/or IL5. Cytokine secretion by T cells can be increased by any of about 5, 10, 15, 20, 25, 30, 35, 40, 45, 50, 55, 60, 65, 70, 75, 80, 85, 90, 95,100,110, 120, 130, 140 or 150% or more (inclusive of all percentages falling in between these values) compared to the amount of cytokine secretion observed in T cells that are not contacted with one or more polypeptide(s) comprising SEQ ID NOs.1-71. Methods to measure cytokine secretion are well known in the art. In some embodiments, the T cell is a cytotoxic T cell, a helper T cell, and/or a γδ T cell.

In some embodiments, contacting the T cell with one or more (such as 1, 2, 3, 4, 5, 6, 7, 8, 9, 10, 11, 12, 13, 14, 15, or more) polypeptide(s) comprising SEQ ID NOs.1-71 (such as one or more of SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO: 12, SEQ ID NO:13, SEQ ID NO:14, SEQ ID NO:15, SEQ ID NO:16, SEQ ID NO: 17, SEQ ID NO: 18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO: 29, SEQ ID NO:30, SEQ ID NO:31, SEQ ID NO:32, SEQ ID NO:33, SEQ ID NO:34, SEQ ID NO: 35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO: 46, SEQ ID NO:47, SEQ ID NO:48, SEQ ID NO:49, SEQ ID NO:50, SEQ ID NO:51, SEQ ID NO: 52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO: 63, SEQ ID NO:64, SEQ ID NO:65, SEQ ID NO:66, SEQ ID NO:67, SEQ ID NO:68, SEQ ID NO: 69, SEQ ID NO:70 and/or SEQ ID NO:71), results in upregulation of one or more surface-expressed activation markers such as, without limitation, CD69, CD134, CD137, CD154, and/or CD25. Surface-expressed activation markers can be upregulated by any of about 5, 10, 15, 20, 25, 30, 35, 40, 45, 50, 55, 60, 65, 70, 75, 80, 85, 90, 95,100,110, 120, 130, 140 or 150% or more (inclusive of all percentages falling in between these values) compared to the amount of surface-expressed activation markers observed in T cells that are not contacted with one or more polypeptide(s) comprising SEQ ID NOs.1-71. Methods to measure surface-expressed activation markers are well known in the art. In some embodiments, the T cell is a cytotoxic T cell, a helper T cell, and/or a γδ T cell.

### Pharmaceutical Composition

mCRAGs of the present invention may be used as—or in the preparation of—a pharmaceutical composition or formulation. Here, the term “pharmaceutical” is used in a broad sense—and covers pharmaceuticals for humans as well as pharmaceuticals for animals (i.e. veterinary applications).

In a preferred embodiment, the pharmaceutical acceptable composition is a medicament.

The pharmaceutical composition can be for therapeutic purposes-which may be curative or palliative or preventative in nature. The pharmaceutical composition may even be for diagnostic purposes.

In a preferred embodiment of the present invention, the medicament is for oral administration.

A pharmaceutically acceptable composition or support may be for example a formulation or support in the form of creams, foams, gels, lotions, and ointments of compressed tablets, tablets, capsules, ointments, suppositories or drinkable solutions.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

Yeasts in general have been shown to be adjuvants in oral administration. Yarrowia, in particular, has been shown to drive the correct IL-12/Th1/interferon gamma path as well as inducing IL-27 leading to CD8 Cytotoxoc T-Lymphocyte synthesis/activation. It also appears to induce IL-17 production by the Th17 cell subset, normally involved in innate immunity of the gut epithelium including wall integrity.

Therefore, in a particular embodiment, the adjuvant is a yeast and, more particularly, Yarrowia.

The pharmaceutical may be in the form of a solution or as a solid-depending on the use and/or the mode of application and/or the mode of administration.

The mCRAGs of the present invention may be used as pharmaceutical ingredients. Here, the composition may be the sole active component, or it may be at least one of a number (i.e. 2 or more) of active components.

The pharmaceutical ingredient may be in the form of a solution or as a solid-depending on the use and/or the mode of application and/or the mode of administration.

The mCRAGs may be used according to the present invention in any suitable form-whether when alone or when present in a combination with other components or ingredients. Likewise, combinations comprising the bacteria of the present invention and other components and/or ingredients (i.e. ingredients-such as food ingredients, functional food ingredients or pharmaceutical ingredients) may be used in any suitable form.

The mCRAGs may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the mCRAGs of the present invention are used in a tablet form-such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid 30 monoglycerides and diglycerides, petroethrai fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the mCRAGs of the present invention may be combined with various sweetening or flavouring agents, colouring matter or dyes, with emulsifying and/or suspending agents and with diluents such as water, propylene glycol and glycerin, and combinations thereof.

The forms may also include gelatin capsules; fibre capsules, fibre tablets etc.; or even fibre beverages.

In one aspect, the mCRAGs according to the present invention may be administered in an aerosol, for example by way of a nasal spray, for instance for administration to the respiratory tract.

### EXAMPLES

### Example 1: Enrichment and Evaluation T-Cell Repertoires Cross-Reactive Between Commensal Microbes and Pathogenic Antigens

In this Example, the cross-reactive potential of microbial peptides having high amino acid sequence homology to known viral pathogen is experimentally determined. In vitro T-cell recall protocols are devised to identify such interactions.

These methods occur in two phases. The first phase involves in vitro stimulation of Peripheral Blood Mononuclear Cells (PBMC) that are obtained from healthy human donors using microbial antigens in the form of heat killed whole cell preparations or synthetic peptide antigens based on amino acid segments of microbial proteins. Memory T-cell responses can occur in donor PBMC samples when the individual donor has had previous immune encounters with the microbial species applied to the in vitro culture system. T-cells that attain activated phenotypes in response to microbial antigens that are consistent with responses known to occur in activation of memory T-cells are isolated from the bulk of non-responsive T-cells in the first phase of the experiment. Responses that are used to differentiate memory cell activation are also used to isolate activated T-cells. These responses include: up regulation of specific surface markers; detection by antibodies and isolation by magnetic separation techniques; or induced proliferation detected by reduced fluorescent intensity of cell tracking dyes followed by isolation by FACS.

Once the subset of T-cells responsive to microbial antigens is obtained, a period of in vitro expansion will occur that is supported through cytokine supplementation. These cytokines can include (IL-2, IL-7, IL-15, IL-4). Further antagonism of T-cell Receptor (TCR) complex and co-stimulatory receptors such as CD28 and CD137 via antibody conjugated polystyrene microspheres is used to mimic stimulatory signals provided by antigen presenting cells. T-cell cultivation methods are used to maintain the in vitro proliferation of T-cells and are continued for 14 days or until enough cells are obtained to seed assays in the second phase of the experiment at which point stimulatory microspheres and cytokines are removed to allow the enriched T-cell lines to return to a resting state.

Rested T-cell lines are re-combined with autologous PBMC cells from the same donor sample used in the initial microbial antigen stimulation at a 1:10 ratio to supply the antigen presentation functions required for T-cell activation in re-stimulation assays. Reserved autologous PBMC are also used to measure the baseline naïve response to peptide antigens applied in the re-stimulation assays.

The following peptides listed in Tables 1 and 2 are used to test cross reactivity in T-cells lines established in methodologies described as phase 1 and include the following.

SARS-CoV-2 peptides: Synthetic peptides corresponding to known immunogenic T-cell epitopes collated the IEDB database which are empirically shown to induce T-cell responses in humans previously infected with a pathogen, in this case SARS-CoV-2.

CRAg (mCRAG) peptide: Microbe derived amino acid sequences with homology to SARS-CoV-2 that maintain HLA binding comparable to the SARS-CoV-2 peptide to which it is homologous to.

CRAg Control peptide: Peptides that are non-homologous to SARS-CoV-2 but are derived from the same microbial protein as the CRAg peptide having ideal HLA binding characteristics to similar HLA alleles as the SARS-CoV-2 peptide and matched CRAg peptide.

TABLE 1
CD8 9 mer peptides

| CD8 | | | |
|---|---|---|---|
| SARS- | | | |
| CoV-2 | | | |
| epitope | SARS-CoV-2 | | |
| ID# | peptide | CRAg peptide | CRAg control peptide |
| 1 | DTDFVNEFY | DVDFRNELY (SEQ | VLDSQRWYY (SEQ ID |
| | (SEQ ID NO: 72) | ID NO: 1) | NO: 118) |
| | | DTDIVNDIY | ITDQINKVY (SEQ ID |
| | | (SEQ ID NO: 2) | NO: 119) |
| 2 | LTDEMIAQY (SEQ | TTNEMIAQY | RADFFSLQL (SEQ ID |
| | ID NO: 73) | (SEQ ID NO: 3) | NO: 120) |
| 3 | ALWEIQQV (SEQ | ALWEIGAVV | FLTGDLGYL (SEQ ID |
| | ID NO: 74) | (SEQ ID NO: 4) | NO: 121) |
| 4 | FIAGLIAIV (SEQ | FISGVIAIV (SEQ | ILVSFISGV (SEQ ID |
| | ID NO: 75) | ID NO: 5) | NO: 122) |
| 5 | FLLNKEMYL | FLLNKEIYR | KMMLGILRA (SEQ ID |
| | (SEQ ID NO: 76) | (SEQ ID NO: 6) | NO: 123) |
| 6 | FLNRFTTTL (SEQ | RLLRFTTTL (SEQ | QLADALAEI (SEQ ID |
| | ID NO: 77) | ID NO: 7) | NO: 124) |
| 7 | GLTVLPPLL (SEQ | GLTILPELL | VVFGGMGSL (SEQ ID |
| | ID NO: 78) | (SEQ ID NO: 8) | NO: 125) |
| 8 | KLWAQCVQL | KLGAQWVQL | SLYDQMLDT (SEQ ID |
| | (SEQ ID NO: 79) | (SEQ ID NO: 9) | NO: 126) |
| 9 | LLYDANYFL | LLYLANYNV | ALDSGELDL (SEQ ID |
| | (SEQ ID NO: 80) | (SEQ ID NO: 10) | NO: 127) |
| | | LMIDVNYFL (SEQ | SLLKYSIPL (SEQ ID |
| | | ID NO: 11) | NO: 128) |
| | | LLYDANDLA | LLFRINMSV (SEQ ID |
| | | (SEQ ID NO: 12) | NO: 129) |
| | | LLYSATSFL | LMMVASNEV (SEQ ID |
| | | (SEQ ID NO: 13) | NO: 130) |
| 10 | RLDKVEAEV | RLDKVDALV | LVANGLIAV (SEQ ID |
| | (SEQ ID NO: 81) | (SEQ ID NO: 14) | NO: 131) |
| 11 | SIIAYTMSL (SEQ | GIDAYTMSL | YLLDVLGEV (SEQ ID |
| | ID NO: 82) | (SEQ ID NO: 15) | NO: 132) |
| | | TILAYSMSL | FTLAIILPV (SEQ ID |
| | | (SEQ ID NO: 16) | NO: 133) |
| | | TIFAYSMSL | GLTGALFFV (SEQ ID |
| | | (SEQ ID NO: 17) | NO: 134) |
| 12 | TLGVLVPHV | TLGVLPPTV | VLAEHSAWV (SEQ ID |
| | (SEQ ID NO: 83) | (SEQ ID NO: 18) | NO: 135) |
| 13 | TLMNVLTLV | TLMNVLSGV | ALNDVNIMV (SEQ ID |
| | (SEQ ID NO: 84) | (SEQ ID NO: 19) | NO: 136) |
| | | SLNNVLTLV | FMPGVLLAV (SEQ ID |
| | | (SEQ ID NO: 20) | NO: 137) |
| 14 | VLNDILSRL (SEQ | SLNDELSRL | TMWIPPDSV (SEQ ID |
| | ID NO: 85) | (SEQ ID NO: 21) | NO: 138) |
| 15 | ALSKGVHFV | ALSKGVRIV | QLMGELSDI (SEQ ID |
| | (SEQ ID NO: 86) | (SEQ ID NO: 22) | NO: 139) |
| | | WIAGVVAIV | VMFSLTGFV (SEQ ID |
| | | (SEQ ID NO: 23) | NO: 140) |
| 16 | FIAGLIAIV (SEQ | FIASVICIV | ALRETVMSA (SEQ ID |
| | ID NO: 87) | (SEQ ID NO: 24) | NO: 141) |
| 17 | GMSRIGMEV | GIIRIGAEV | ILAEKPGVV (SEQ ID |
| | (SEQ ID NO: 88) | (SEQ ID NO: 25) | NO: 142) |
| | | RLNEESSNL | FVRGYTSTV (SEQ ID |
| | | (SEQ ID NO: 26) | NO: 143) |
| 18 | RLNEVAKNL | RLMEVTKNA | ILAGMHIGL (SEQ ID |
| | (SEQ ID NO: 89) | (SEQ ID NO: 27) | NO: 144) |
| | | RLSEVENNL | LLSKHVKSV (SEQ ID |
| | | (SEQ ID NO: 28) | NO: 145) |
| 19 | YLATALLTL | YLATVVLTG | MIAGRSTAM (SEQ ID |
| | (SEQ ID NO: 90) | (SEQ ID NO: 29) | NO: 146) |
| 20 | LALLLLDRL | QALLFLDRL | GVDNRPVAV (SEQ ID |
| | (SEQ ID NO: 91) | (SEQ ID NO: 30) | NO: 147) |
| 21 | LLLDRLNQL | LILDHLHQL | LTIAILTAL (SEQ ID |
| | (SEQ ID NO: 92) | (SEQ ID NO: 31) | NO: 148) |
| | | ILLDGLQQL | ALIEEGTPV (SEQ ID |
| | | (SEQ ID NO: 32) | NO: 149) |
| 22 | LQLGFSTGV | LQLGFSRGL | LQGSWLGPI (SEQ ID |
| | (SEQ ID NO: 93) | (SEQ ID NO: 33) | NO: 150) |
| | | LQLGASTGV | FAIAIGLYV (SEQ ID |
| | | (SEQ ID NO: 34) | NO: 151) |
| 23 | LVLSVNPYV | LVLSVNGYL | YLLGLLPAI (SEQ ID |
| | (SEQ ID NO: 94) | (SEQ ID NO: 35) | NO: 152) |
| 24 | ALRANSAVK | ALQANAAVK | IIGASGSGK (SEQ ID |
| | (SEQ ID NO: 95) | (SEQ ID NO: 36) | NO: 153) |
| 25 | ASAFFGMSR | AVAFFLMSR | RQQAIFFRK (SEQ ID |
| | (SEQ ID NO: 96) | (SEQ ID NO: 37) | NO: 154) |
| 26 | GTITVEELK (SEQ | GTITVHELR | SAIRIFLTR (SEQ ID |
| | ID NO: 97 | (SEQ ID NO: 38) | NO: 155) |
| 27 | KTFPPTEPK (SEQ | ETFDPTKPK | YTDNVPLLK (SEQ ID |
| | ID NO: 98) | (SEQ ID NO: 39) | NO: 189) |
| | | KTPAPTTPK | GLANFKFYR (SEQ ID |
| | | (SEQ ID NO: 40) | NO: 156) |
| 28 | VYIGDPAQL | VYLGDQARL | HYPILRPLI (SEQ ID |
| | (SEQ ID NO: 99) | (SEQ ID NO: 41) | NO: 157) |
| 29 | YYQLYSTQL | YYQLYTGSL | PYTVFVLVF (SEQ ID |
| | (SEQ ID NO: 100) | (SEQ ID NO: 42) | NO: 158) |
| | | YYQRHSTRL | HWQASTANF (SEQ ID |
| | | (SEQ ID NO: 43) | NO: 159) |
| 30 | FPRGQGVPI (SEQ | KPLGGGVPI (SEQ | VPMSYEAAL (SEQ ID |
| | ID NO: 188) | ID NO: 44) | NO: 160) |
| | | FPRGRIVEI | APGRETASV (SEQ ID |
| | | (SEQ ID NO: 45) | NO: 161) |
| | | MPQWQGVPI | MPTYPAATM (SEQ ID |
| | | (SEQ ID NO: 46) | NO: 162) |
| | | FVRGQGVTV | IPGKPVSEV (SEQ ID |
| | | (SEQ ID NO: 47) | NO: 163) |
| 31 | IPRRNVATL (SEQ | IPRQNVVTV | RIRGVSKSF (SEQ ID |
| | ID NO: 101) | (SEQ ID NO: 48) | NO: 164) |
| 32 | SPRRARSVA | SVRRARAVA | LPAHGESVL (SEQ ID |
| | (SEQ ID NO: 102) | (SEQ ID NO: 49) | NO: 165) |
| 33 | GEAANFCAL | VEAANDCAL | SENRGLITV (SEQ ID |
| | (SEQ ID NO: 103) | (SEQ ID NO: 50) | NO: 166) |
| | | GQAANFFAL | GESISNPAL (SEQ ID |
| | | (SEQ ID NO: 51) | NO: 167) |
| 34 | GETLPTEVL (SEQ | GETLPIEHL | RETETGLIL (SEQ ID |
| | ID NO: 104) | (SEQ ID NO: 52) | NO: 168) |
| | | GEALPTLVL | WEDMAKSGI (SEQ ID |
| | | (SEQ ID NO: 53) | NO: 169) |
| 35 | LEPLVDLPI (SEQ | LERLADLPI | VEDQDHPSV (SEQ ID |
| | ID NO: 105) | (SEQ ID NO: 54) | NO: 170) |
| 36 | MEVTPSGTW | MEQTKDGTW | KEALDAIHF (SEQ ID |
| | (SEQ ID NO: 106) | (SEQ ID NO: 55) | NO: 171) |
| 37 | CTDDNALAY | VLDDNHLAY | ISEIEDLSA (SEQ ID |
| | (SEQ ID NO: 107) | (SEQ ID NO: 56) | NO: 172) |
| | | ITDENAPAY | RTDNWFLLA (SEQ ID |
| | | (SEQ ID NO: 57) | NO: 173) |
| | | STDDRAAAY | CTGHGNGLY (SEQ ID |
| | | (SEQ ID NO: 58) | NO: 174) |

TABLE 2
CD4 15mer peptides

| CD4 SARS- | | | |
|---|---|---|---|
| CoV-2 | | | |
| epitope ID# | SARS-CoV-2 peptide | CRAg peptide | CRAg control peptide |
| 1 | IILFLALITLATCEL | IIYFLALITVSTSLL | SFSTAFFTAVSALST |
| | (SEQ ID NO: 108) | (SEQ ID NO: 59) | (SEQ ID NO: 175) |
| 2 | LMIERFVSLAIDAYP | LGIERTVSLARRAWG | KVQLLTLFSTMLGIE |
| | (SEQ ID NO: 109) | (SEQ ID NO: 60) | (SEQ ID NO: 176) |
| 3 | AADLDDESKQLQQS | EFGLDDFLAQLQQVR | LQQVRKLGSMKSLL |
| | M (SEQ ID NO: 110) | (SEQ ID NO: 61) | G (SEQ ID NO: 177) |
| | | AADGWDLDSQLEQA | YGDRVILDDVTLSFL |
| | | M | (SEQ ID NO: 178) |
| | | (SEQ ID NO: 62) | |
| | | SFGLDDFLDQLQQVR | YDTVIIDTAGRLGVD |
| | | (SEQ ID NO: 63) | (SEQ ID NO: 179) |
| | | PADLDDIKTELQQEG | QNRYRTLLPGATYR |
| | | (SEQ ID NO: 64) | Q (SEQ ID NO: 180) |
| 4 | DAALALLLLDRLNQ | DIELAALELDRLHQA | YDVLQLLKGFGTYI |
| | L (SEQ ID NO: 111) | (SEQ ID NO: 65) | H (SEQ ID NO: 181) |
| 5 | KVTFFPDLNGDVVAI | KPSFFPDLLEDVRAQ | YFYDQLAAASSVPL |
| | (SEQ ID NO: 112) | (SEQ ID NO: 66) | L (SEQ ID NO: 182) |
| 6 | TFKCYGVSPTKLND | VFKDYQVSHTKMRD | RHQYVLDFSNPAVV |
| | L (SEQ ID NO: 113) | L | N (SEQ ID NO: 183) |
| | | (SEQ ID NO: 67) | |
| 7 | MFVFLVLLPLVSS | TFLIL VLIPLTSS | GGSRSLLAMGNLLA |
| | (SEQ ID NO: 114) | (SEQ ID NO:68) | P (SEQ ID NO:184) |
| 8 | EFYAYLRKHFSMMI | EFYAYISTGVIMMIL | EVMKPAYILMLGRV |
| | L (SEQ ID NO: 115) | (SEQ ID NO: 69) | V (SEQ ID NO: 185) |
| 9 | NKDGIIWVATEGAL | NNSKIIAVATGGSLN | IILGVLLILLLAYLS |
| | N (SEQ ID NO: 116) | (SEQ ID NO: 70) | (SEQ ID NO: 186) |
| 10 | NLLLQYGSFCTQLN | VLVLMIGSFCTILNA | SFIAPNFATLLAGRL |
| | R (SEQ ID NO: 117) | (SEQ ID NO: 71) | (SEQ ID NO: 187) |

Assays to determine cross reactivity are conducted using flow cytometry-based assays that measure activation signals that include intracellular accumulation of cytokines, surface levels of activation induced markers of T-cell activation, or proliferation in response to peptide stimulation.

### Example 2: Amplification of T-cells Reactive to Probiotic Derived CRAG Peptides Increases the Percentage of SARS-CoV-2 Reactive TCR Clonotypes In Healthy Human Donor PBMC

In order to demonstrate that CRAG peptides are capable of cross activating T-cell receptors (TCR) reactive towards SARS-Cov-2 peptide antigens, a 2-step ex-vivo assay was conducted using cryopreserved PBMC preparations from healthy donors. The PBMC were cultured in media containing pooled peptides where the amino acid sequence of the peptides originated from proteins expressed by the probiotic organisms. In one peptide pool the probiotic sequences share a high degree of amino acid sequence homology with SARS-CoV-2 antigens known to illicit recall responses in T-cells collected from convalescent Covid-19 patients. The second pool consisted of a set of matched peptides from the same probiotic protein but lacking homologous sequence identity to SARS-CoV-2. Additionally, each paired CRAG and Control peptide were screened for sequence motifs that would predict their ability to bind with similar affinity to the same HLA alleles as the parent SARS-CoV-2 antigen. This was to aimed to maximize the probability that the same sets of peptides within each peptide pool may be recognized by individual donor antigen presenting cells despite high variability in HLA allele genetics in the donor population. Thus, the CRAG and matched control peptide pools were incubated in separate wells from each donor over a 2-week incubation period to support the proliferation of TCR specific clonotypes in each pool. In the final 48 hours of the protocol the cytokine and antigen supplements supporting antigen induced activation and proliferation were removed to allow T-cells to return to a resting state.

In the second phase of the protocol, the proportion of T-cells in the CRAG and Control peptide stimulated donor fractions were re-stimulated with various peptide pools and control treatments for 16 hours and analyzed for CD4⁺ T-cell intracellular IFN-γ production by flow cytometry. Each ex-vivo amplified fraction was re-stimulated with CRAG, Control, and SARS-CoV-2 peptide pools. CRAG and Control fractions from the same donor were treated as paired samples to compare the difference in % IFN-γ positive CD4 cells upon re-stimulation.

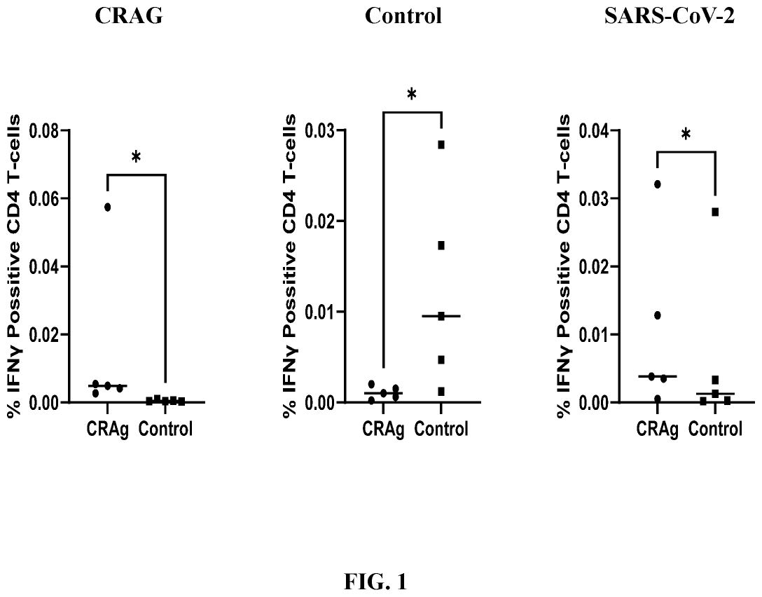

As expected, CRAG fractions contained more CRAG reactive CD4 T-Cells than Control fractions from the same donor and conversely Control fractions contained more Control peptide reactive than CRAG fractions. As shown in FIG. 1, in response to SARS-CoV-2 peptide re-stimulation, there was a higher percentage of IFN-γ in the CRAG fractions indicating that CRAG reactive clonotypes are likely contributing to the signal generated in response to the SARS-CoV-2 peptide. This result indicates that, at the molecular level, the degree of sequence homology that can be found in amino acid segments contained within probiotic proteins are sufficient to induce activation of T-cells known to be involved in the immune response toward SARS-CoV-2.

1. A method for stimulating a T cell, comprising contacting the T cell with one or more polypeptide(s) comprising SEQ ID NOs.1-71, wherein stimulating the T cell results in one or more of i) increased T cell proliferation; ii) secretion of cytokines; and iii) upregulation of one or more surface-expressed activation markers.

2. The method of claim 1, wherein the cytokine is one or more cytokine selected from the group consisting of IFNγ, TNFα, IL2, IL17, IL22, IL4, and IL5.

3. The method of claim 1 or claim 2, wherein the surface-expressed activation marker is one or more marker selected from the group consisting of CD69, CD134, CD137, CD154, and CD25.

4. The method of any one of claims 1-3, wherein the T-cell is a cytotoxic T cell, a helper T cell, and/or a γδ T cell.

5. The method of any one of claims 1-4, wherein said one or more polypeptides are expressed in a cell.


SEQUENCE LISTING (189 sequences)

SEQ ID NO: 1 - protein, 9 aa, synthetic construct
DVDFRNELY

SEQ ID NO: 2 - protein, 9 aa, synthetic construct
DTDIVNDIY

SEQ ID NO: 3 - protein, 9 aa, synthetic construct
TTNEMIAQY

SEQ ID NO: 4 - protein, 9 aa, synthetic construct
ALWEIGAVV

SEQ ID NO: 5 - protein, 9 aa, synthetic construct
FISGVIAIV

SEQ ID NO: 6 - protein, 9 aa, synthetic construct
FLLNKEIYR

SEQ ID NO: 7 - protein, 9 aa, synthetic construct
RLLRFTTTL

SEQ ID NO: 8 - protein, 9 aa, synthetic construct
GLTILPELL

SEQ ID NO: 9 - protein, 9 aa, synthetic construct
KLGAQWVQL

SEQ ID NO: 10 - protein, 9 aa, synthetic construct
LLYLANYNV

SEQ ID NO: 11 - protein, 9 aa, synthetic construct
LMIDVNYFL

SEQ ID NO: 12 - protein, 9 aa, synthetic construct
LLYDANDLA

SEQ ID NO: 13 - protein, 9 aa, synthetic construct
LLYSATSFL

SEQ ID NO: 14 - protein, 9 aa, synthetic construct
RLDKVDALV

SEQ ID NO: 15 - protein, 9 aa, synthetic construct
GIDAYTMSL

SEQ ID NO: 16 - protein, 9 aa, synthetic construct
TILAYSMSL

SEQ ID NO: 17 - protein, 9 aa, synthetic construct
TIFAYSMSL

SEQ ID NO: 18 - protein, 9 aa, synthetic construct
TLGVLPPTV

SEQ ID NO: 19 - protein, 9 aa, synthetic construct
TLMNVLSGV

SEQ ID NO: 20 - protein, 9 aa, synthetic construct
SLNNVLTLV

SEQ ID NO: 21 - protein, 9 aa, synthetic construct
SLNDELSRL

SEQ ID NO: 22 - protein, 9 aa, synthetic construct
ALSKGVRIV

SEQ ID NO: 23 - protein, 9 aa, synthetic construct
WIAGVVAIV

SEQ ID NO: 24 - protein, 9 aa, synthetic construct
FIASVICIV

SEQ ID NO: 25 - protein, 9 aa, synthetic construct
GIIRIGAEV

SEQ ID NO: 26 - protein, 9 aa, synthetic construct
RLNEESSNL

SEQ ID NO: 27 - protein, 9 aa, synthetic construct
RLMEVTKNA

SEQ ID NO: 28 - protein, 9 aa, synthetic construct
RLSEVFNNL

SEQ ID NO: 29 - protein, 9 aa, synthetic construct
YLATVVLTG

SEQ ID NO: 30 - protein, 9 aa, synthetic construct
QALLFLDRL

SEQ ID NO: 31 - protein, 9 aa, synthetic construct
LILDHLHQL

SEQ ID NO: 32 - protein, 9 aa, synthetic construct
ILLDGLQQL

SEQ ID NO: 33 - protein, 9 aa, synthetic construct
LQLGFSRGL

SEQ ID NO: 34 - protein, 9 aa, synthetic construct
LQLGASTGV

SEQ ID NO: 35 - protein, 9 aa, synthetic construct
LVLSVNGYL

SEQ ID NO: 36 - protein, 9 aa, synthetic construct
ALQANAAVK

SEQ ID NO: 37 - protein, 9 aa, synthetic construct
AVAFFLMSR

SEQ ID NO: 38 - protein, 9 aa, synthetic construct
GTITVHELR

SEQ ID NO: 39 - protein, 9 aa, synthetic construct
ETFDPTKPK

SEQ ID NO: 40 - protein, 9 aa, synthetic construct
KTPAPTTPK

SEQ ID NO: 41 - protein, 9 aa, synthetic construct
VYLGDQARL

SEQ ID NO: 42 - protein, 9 aa, synthetic construct
YYQLYTGSL

SEQ ID NO: 43 - protein, 9 aa, synthetic construct
YYQRHSTRL

SEQ ID NO: 44 - protein, 9 aa, synthetic construct
KPLGGGVPI

SEQ ID NO: 45 - protein, 9 aa, synthetic construct
FPRGRIVEI

SEQ ID NO: 46 - protein, 9 aa, synthetic construct
MPQWQGVPI

SEQ ID NO: 47 - protein, 9 aa, synthetic construct
FVRGQGVTV

SEQ ID NO: 48 - protein, 9 aa, synthetic construct
IPRQNVVTV

SEQ ID NO: 49 - protein, 9 aa, synthetic construct
SVRRARAVA

SEQ ID NO: 50 - protein, 9 aa, synthetic construct
VEAANDCAL

SEQ ID NO: 51 - protein, 9 aa, synthetic construct
GQAANFFAL

SEQ ID NO: 52 - protein, 9 aa, synthetic construct
GETLPIEHL

SEQ ID NO: 53 - protein, 9 aa, synthetic construct
GEALPTLVL

SEQ ID NO: 54 - protein, 9 aa, synthetic construct
LERLADLPI

SEQ ID NO: 55 - protein, 9 aa, synthetic construct
MEQTKDGTW

SEQ ID NO: 56 - protein, 9 aa, synthetic construct
VLDDNHLAY

SEQ ID NO: 57 - protein, 9 aa, synthetic construct
ITDENAPAY

SEQ ID NO: 58 - protein, 9 aa, synthetic construct
STDDRAAAY

SEQ ID NO: 59 - protein, 15 aa, synthetic construct
IIYFLALITVSTSLL

SEQ ID NO: 60 - protein, 15 aa, synthetic construct
LGIERTVSLARRAWG

SEQ ID NO: 61 - protein, 15 aa, synthetic construct
EFGLDDFLAQLQQVR

SEQ ID NO: 62 - protein, 15 aa, synthetic construct
AADGWDLDSQLEQAM

SEQ ID NO: 63 - protein, 15 aa, synthetic construct
SFGLDDFLDQLQQVR

SEQ ID NO: 64 - protein, 15 aa, synthetic construct
PADLDDIKTELQQEG

SEQ ID NO: 65 - protein, 15 aa, synthetic construct
DIELAALELDRLHQA

SEQ ID NO: 66 - protein, 15 aa, synthetic construct
KPSFFPDLLEDVRAQ

SEQ ID NO: 67 - protein, 15 aa, synthetic construct
VFKDYQVSHTKMRDL

SEQ ID NO: 68 - protein, 13 aa, synthetic construct
TFLILVLIPLTSS

SEQ ID NO: 69 - protein, 15 aa, synthetic construct
EFYAYISTGVIMMIL

SEQ ID NO: 70 - protein, 15 aa, synthetic construct
NNSKIIAVATGGSLN

SEQ ID NO: 71 - protein, 15 aa, synthetic construct
VLVLMIGSFCTILNA

SEQ ID NO: 72 - protein, 9 aa, synthetic construct
DTDFVNEFY

SEQ ID NO: 73 - protein, 9 aa, synthetic construct
LTDEMIAQY

SEQ ID NO: 74 - protein, 8 aa, synthetic construct
ALWEIQQV

SEQ ID NO: 75 - protein, 9 aa, synthetic construct
FIAGLIAIV

SEQ ID NO: 76 - protein, 9 aa, synthetic construct
FLLNKEMYL

SEQ ID NO: 77 - protein, 9 aa, synthetic construct
FLNRFTTTL

SEQ ID NO: 78 - protein, 9 aa, synthetic construct
GLTVLPPLL

SEQ ID NO: 79 - protein, 9 aa, synthetic construct
KLWAQCVQL

SEQ ID NO: 80 - protein, 9 aa, synthetic construct
LLYDANYFL

SEQ ID NO: 81 - protein, 9 aa, synthetic construct
RLDKVEAEV

SEQ ID NO: 82 - protein, 9 aa, synthetic construct
SIIAYTMSL

SEQ ID NO: 83 - protein, 9 aa, synthetic construct
TLGVLVPHV

SEQ ID NO: 84 - protein, 9 aa, synthetic construct
TLMNVLTLV

SEQ ID NO: 85 - protein, 9 aa, synthetic construct
VLNDILSRL

SEQ ID NO: 86 - protein, 9 aa, synthetic construct
ALSKGVHFV

SEQ ID NO: 87 - protein, 9 aa, synthetic construct
FIAGLIAIV

SEQ ID NO: 88 - protein, 9 aa, synthetic construct
GMSRIGMEV

SEQ ID NO: 89 - protein, 9 aa, synthetic construct
RLNEVAKNL

SEQ ID NO: 90 - protein, 9 aa, synthetic construct
YLATALLTL

SEQ ID NO: 91 - protein, 9 aa, synthetic construct
LALLLLDRL

SEQ ID NO: 92 - protein, 9 aa, synthetic construct
LLLDRLNQL

SEQ ID NO: 93 - protein, 9 aa, synthetic construct
LQLGFSTGV

SEQ ID NO: 94 - protein, 9 aa, synthetic construct
LVLSVNPYV

SEQ ID NO: 95 - protein, 9 aa, synthetic construct
ALRANSAVK

SEQ ID NO: 96 - protein, 9 aa, synthetic construct
ASAFFGMSR

SEQ ID NO: 97 - protein, 9 aa, synthetic construct
GTITVEELK

SEQ ID NO: 98 - protein, 9 aa, synthetic construct
KTFPPTEPK

SEQ ID NO: 99 - protein, 9 aa, synthetic construct
VYIGDPAQL

SEQ ID NO: 100 - protein, 9 aa, synthetic construct
YYQLYSTQL

SEQ ID NO: 101 - protein, 9 aa, synthetic construct
IPRRNVATL

SEQ ID NO: 102 - protein, 9 aa, synthetic construct
SPRRARSVA

SEQ ID NO: 103 - protein, 9 aa, synthetic construct
GEAANFCAL

SEQ ID NO: 104 - protein, 9 aa, synthetic construct
GETLPTEVL

SEQ ID NO: 105 - protein, 9 aa, synthetic construct
LEPLVDLPI

SEQ ID NO: 106 - protein, 9 aa, synthetic construct
MEVTPSGTW

SEQ ID NO: 107 - protein, 9 aa, synthetic construct
CTDDNALAY

SEQ ID NO: 108 - protein, 15 aa, synthetic construct
IILFLALITLATCEL

SEQ ID NO: 109 - protein, 15 aa, synthetic construct
LMIERFVSLAIDAYP

SEQ ID NO: 110 - protein, 15 aa, synthetic construct
AADLDDFSKQLQQSM

SEQ ID NO: 111 - protein, 15 aa, synthetic construct
DAALALLLLDRLNQL

SEQ ID NO: 112 - protein, 15 aa, synthetic construct
KVTFFPDLNGDVVAI

SEQ ID NO: 113 - protein, 15 aa, synthetic construct
TFKCYGVSPTKLNDL

SEQ ID NO: 114 - protein, 13 aa, synthetic construct
MFVFLVLLPLVSS

SEQ ID NO: 115 - protein, 15 aa, synthetic construct
EFYAYLRKHFSMMIL

SEQ ID NO: 116 - protein, 15 aa, synthetic construct
NKDGIIWVATEGALN

SEQ ID NO: 117 - protein, 15 aa, synthetic construct
NLLLQYGSFCTQLNR

SEQ ID NO: 118 - protein, 9 aa, synthetic construct
VLDSQRWYY

SEQ ID NO: 119 - protein, 9 aa, synthetic construct
ITDQINKVY

SEQ ID NO: 120 - protein, 9 aa, synthetic construct
RADFFSLQL

SEQ ID NO: 121 - protein, 9 aa, synthetic construct
FLTGDLGYL

SEQ ID NO: 122 - protein, 9 aa, synthetic construct
ILVSFISGV

SEQ ID NO: 123 - protein, 9 aa, synthetic construct
KMMLGILRA

SEQ ID NO: 124 - protein, 9 aa, synthetic construct
QLADALAEI

SEQ ID NO: 125 - protein, 9 aa, synthetic construct
VVFGGMGSL

SEQ ID NO: 126 - protein, 9 aa, synthetic construct
SLYDQMLDT

SEQ ID NO: 127 - protein, 9 aa, synthetic construct
ALDSGELDL

SEQ ID NO: 128 - protein, 9 aa, synthetic construct
SLLKYSIPL

SEQ ID NO: 129 - protein, 9 aa, synthetic construct
LLFRINMSV

SEQ ID NO: 130 - protein, 9 aa, synthetic construct
LMMVASNEV

SEQ ID NO: 131 - protein, 9 aa, synthetic construct
LVANGLIAV

SEQ ID NO: 132 - protein, 9 aa, synthetic construct
YLLDVLGEV

SEQ ID NO: 133 - protein, 9 aa, synthetic construct
FTLAIILPV

SEQ ID NO: 134 - protein, 9 aa, synthetic construct
GLTGALFFV

SEQ ID NO: 135 - protein, 9 aa, synthetic construct
VLAEHSAWV

SEQ ID NO: 136 - protein, 9 aa, synthetic construct
ALNDVNIMV

SEQ ID NO: 137 - protein, 9 aa, synthetic construct
FMPGVLLAV

SEQ ID NO: 138 - protein, 9 aa, synthetic construct
TMWIPPDSV

SEQ ID NO: 139 - protein, 9 aa, synthetic construct
QLMGELSDI

SEQ ID NO: 140 - protein, 9 aa, synthetic construct
VMFSLTGFV

SEQ ID NO: 141 - protein, 9 aa, synthetic construct
ALRETVMSA

SEQ ID NO: 142 - protein, 9 aa, synthetic construct
ILAEKPGVV

SEQ ID NO: 143 - protein, 9 aa, synthetic construct
FVRGYTSTV

SEQ ID NO: 144 - protein, 9 aa, synthetic construct
ILAGMHIGL

SEQ ID NO: 145 - protein, 9 aa, synthetic construct
LLSKHVKSV

SEQ ID NO: 146 - protein, 9 aa, synthetic construct
MIAGRSTAM

SEQ ID NO: 147 - protein, 9 aa, synthetic construct
GVDNRPVAV

SEQ ID NO: 148 - protein, 9 aa, synthetic construct
LTIAILTAL

SEQ ID NO: 149 - protein, 9 aa, synthetic construct
ALIEEGTPV

SEQ ID NO: 150 - protein, 9 aa, synthetic construct
LQGSWLGPI

SEQ ID NO: 151 - protein, 9 aa, synthetic construct
FAIAIGLYV

SEQ ID NO: 152 - protein, 9 aa, synthetic construct
YLLGLLPAI

SEQ ID NO: 153 - protein, 9 aa, synthetic construct
IIGASGSGK

SEQ ID NO: 154 - protein, 9 aa, synthetic construct
RQQAIFFRK

SEQ ID NO: 155 - protein, 9 aa, synthetic construct
SAIRIFLTR

SEQ ID NO: 156 - protein, 9 aa, synthetic construct
GLANFKFYR

SEQ ID NO: 157 - protein, 9 aa, synthetic construct
HYPILRPLI

SEQ ID NO: 158 - protein, 9 aa, synthetic construct
PYTVFVLVF

SEQ ID NO: 159 - protein, 9 aa, synthetic construct
HWQASTANF

SEQ ID NO: 160 - protein, 9 aa, synthetic construct
VPMSYEAAL

SEQ ID NO: 161 - protein, 9 aa, synthetic construct
APGRETASV

SEQ ID NO: 162 - protein, 9 aa, synthetic construct
MPTYPAATM

SEQ ID NO: 163 - protein, 9 aa, synthetic construct
IPGKPVSEV

SEQ ID NO: 164 - protein, 9 aa, synthetic construct
RIRGVSKSF

SEQ ID NO: 165 - protein, 9 aa, synthetic construct
LPAHGESVL

SEQ ID NO: 166 - protein, 9 aa, synthetic construct
SENRGLITV

SEQ ID NO: 167 - protein, 9 aa, synthetic construct
GESISNPAL

SEQ ID NO: 168 - protein, 9 aa, synthetic construct
RETETGLIL

SEQ ID NO: 169 - protein, 9 aa, synthetic construct
WEDMAKSGI

SEQ ID NO: 170 - protein, 9 aa, synthetic construct
VEDQDHPSV

SEQ ID NO: 171 - protein, 9 aa, synthetic construct
KEALDAIHF

SEQ ID NO: 172 - protein, 9 aa, synthetic construct
ISEIEDLSA

SEQ ID NO: 173 - protein, 9 aa, synthetic construct
RTDNWFLLA

SEQ ID NO: 174 - protein, 9 aa, synthetic construct
CTGHGNGLY

SEQ ID NO: 175 - protein, 15 aa, synthetic construct
SFSTAFFTAVSALST

SEQ ID NO: 176 - protein, 15 aa, synthetic construct
KVQLLTLFSTMLGIE

SEQ ID NO: 177 - protein, 15 aa, synthetic construct
LQQVRKLGSMKSLLG

SEQ ID NO: 178 - protein, 15 aa, synthetic construct
YGDRVILDDVTLSFL

SEQ ID NO: 179 - protein, 15 aa, synthetic construct
YDTVIIDTAGRLGVD

SEQ ID NO: 180 - protein, 15 aa, synthetic construct
QNRYRTLLPGATYRQ

SEQ ID NO: 181 - protein, 15 aa, synthetic construct
YDVLQLLKGFGTYIH

SEQ ID NO: 182 - protein, 15 aa, synthetic construct
YFYDQLAAASSVPLL

SEQ ID NO: 183 - protein, 15 aa, synthetic construct
RHQYVLDFSNPAVVN

SEQ ID NO: 184 - protein, 15 aa, synthetic construct
GGSRSLLAMGNLLAP

SEQ ID NO: 185 - protein, 15 aa, synthetic construct
EVMKPAYILMLGRVV

SEQ ID NO: 186 - protein, 15 aa, synthetic construct
IILGVLLILLLAYLS

SEQ ID NO: 187 - protein, 15 aa, synthetic construct
SFIAPNFATLLAGRL

SEQ ID NO: 188 - protein, 9 aa, synthetic construct
FPRGQGVPI

SEQ ID NO: 189 - protein, 9 aa, synthetic construct
YTDNVPLLK